(19) United States
(12) Patent Application Publication
(10) Pub. No.: US 2022/0379096 A1
(43) Pub. Date: Dec. 1, 2022

(54) GUIDE WIRE

(71) Applicant: GUNZE LIMITED, Kyoto (JP)

(72) Inventors: Shinji HARUYAMA, Aichi (JP); Ten YAMAGATA, Aichi (JP)

(21) Appl. No.: 17/883,251

(22) Filed: Aug. 8, 2022

Related U.S. Application Data
(63) Continuation of application No. 16/967,862, filed on Nov. 12, 2020.

(30) Foreign Application Priority Data
Feb. 9, 2018 (JP) 2018-021604

(51) Int. Cl. A61M 25/09

(52) U.S. Cl. CPC A61M 25/09; A61M 2025/09075; A61M 2025/09133; A61M 2205/584

(57) ABSTRACT

Provided is a medical guidewire that ensures adhesion of a coating layer and exhibits excellent smoothness. A medical guidewire including a wire body that is flexible and long, an intermediate layer that has at least one layer and covers a surface of the wire body, and an outermost layer that covers a surface of the intermediate layer, in which the intermediate layer is colored by including a pigment, and a concentration of the pigment is wt % or more and wt % or less with respect to the intermediate layer as a whole.

### TECHNICAL FIELD

The present invention relates to a medical guidewire.

### BACKGROUND ART

Conventionally, various medical guidewires used in a medical setting have been known. For example, as disclosed in Patent Document 1, there is known, as a medical guidewire used endoscopically, a guidewire coated with a fluororesin tube having a spiral pattern colored in multiple colors in order to recognize a movement of the guidewire through an endoscopic fiberscope while ensuring smoothness within a catheter.

### SUMMARY OF THE INVENTION

### Problems to be Solved by the Invention

Although the guidewire coated with the fluororesin tube colored in multiple colors has excellent visibility in endoscopic procedures, when the guidewire is used for a guidewire that is passed through a puncture needle in an ultrasonic endoscopic paracentesis, which has rapidly spread in recent years, a part of the fluororesin tube peels off and falls off due to contact with a distal end of the hollow puncture needle because the fluororesin tube is not chemically bonded to a metal wire rod of the guidewire. Thus, there is a problem that such a guidewire is difficult to use as a guidewire for passing through a puncture needle.

There is also known a guidewire formed by coating a metal wire rod as a core material of the guidewire with a resin underlayer and a fluororesin layer by a coating method. However, such a guidewire does not consider sliding with a metal member having a sharp distal end such as a puncture needle, and the fluororesin layer coated on the resin underlayer may peel off and fall off due to contact with the distal end of the hollow puncture needle. Thus, there is a concern that such a guidewire is difficult to use as a guidewire used for passing through a puncture needle.

Due to such a problem, a metal wire without a coating of a fluororesin tube or a coating layer is used for a guidewire for passing through a puncture needle in an ultrasonic endoscopic paracentesis. However, there is a problem that the metal wire is poor in smoothness and is difficult to insert into a hollow puncture needle.

The present invention has been made to solve these problems, and an object of the present invention is to provide a medical guidewire that ensures adhesion of a coating layer and exhibits excellent smoothness.

### Means for Solving the Problems

The above-mentioned object of the present invention is achieved by a medical guidewire including a wire body that is flexible and long, an intermediate layer that has at least one layer and covers a surface of the wire body, and an outermost layer that covers a surface of the intermediate layer, in which the intermediate layer is colored by including a pigment, and the pigment has a concentration of 50 wt % or more and 90 wt % or less with respect to the intermediate layer as a whole.

In this medical guidewire, the concentration of the pigment is preferably 55 wt % or more and 85 wt % or less with respect to the intermediate layer as a whole.

Further, the intermediate layer can include a first region including a first pigment, and a second region including a second pigment having a color different from a color of the first pigment.

An average particle diameter of the pigment is preferably in a range of 0.05 μm or more and 2 μm or less. A thickness of the intermediate layer is preferably in a range of 1 μm or more and 30 μm or less.

Further, the intermediate layer preferably includes a binder resin including a polyimide resin. Further, the outermost layer is preferably formed of a fluororesin material. The outermost layer is preferably fused to the surface of the intermediate layer.

### Effects of the Invention

The present invention can provide the medical guidewire that ensures adhesion of the coating layer and exhibits excellent smoothness.

### BRIEF DESCRIPTION OF THE DRAWINGS

FIG. 1 is an enlarged schematic sectional view of a configuration of a main part of a medical guidewire according to an embodiment of the present invention.

FIG. 2(a) is an enlarged plan view of a main part showing a modification of the medical guidewire according to FIG. 1, and FIG. 2(b) is an enlarged sectional view of a main part taken along line A-A.

FIG. 3(a) is an enlarged plan view of a main part showing another modification of the medical guidewire according to FIG. 1, and FIG. 3(b) is an enlarged sectional view of a main part taken along line B-B.

FIGS. 4(a) and 4(b) are enlarged plan views of a main part showing still another modification of the medical guidewire according to FIG. 1.

FIG. 5 is an enlarged schematic sectional view of a configuration of a main part showing a modification of the medical guidewire according to FIG. 1.

### EMBODIMENTS OF THE INVENTION

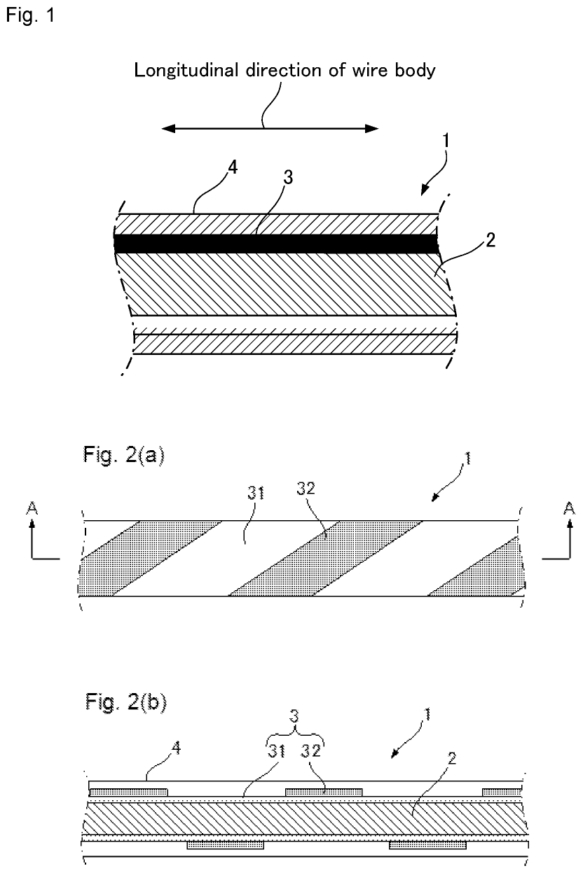

Hereinafter, a medical guidewire 1 according to an embodiment of the present invention will be described with reference to the accompanying drawings. Each drawing is partially enlarged or reduced to facilitate understanding of configurations. FIG. 1 is an enlarged schematic sectional view of a main part of the medical guidewire 1 according to the embodiment of the present invention. The medical guidewire 1 according to the present invention is, for example, a guidewire passed through a hollow puncture needle in an ultrasonic endoscopic paracentesis, or a guidewire used for inserting into a catheter, and the medical guidewire 1 includes a wire body 2, an intermediate layer 3, and an outermost layer 4.

The wire body 2 is a flexible and long linear member. The wire body 2 can be configured by various conventional materials used as a core material for a medical guidewire, but is preferably configured by a metal material. For example, a stainless steel (for example, every SUS type of SUS304, SUS303, SUS316, SUS316L, SUS316J1, SUS316J1L, SUS405, SUS430, SUS434, SUS444, SUS429, SUS430F, or SUS302) can be used. When a stainless steel is used as the material of the wire body 2, the medical guidewire 1 can obtain more excellent pushing property and torque transmissibility.

Further, as the material of the wire body 2, an alloy showing pseudoelasticity (including a superelastic alloy) can be used. In particular, when the wire body 2 is configured by a superelastic alloy, the medical guidewire 1 can obtain sufficient flexibility and resilience for bending as a whole, improved followability for complicated curving and bending, and more excellent operability. Furthermore, even when repeatedly curved and bent, the wire body 2 does not have a bending tendency due to its resilience, and thus can prevent a degradation of the operability due to a bending tendency of the wire body 2 during use of the medical guidewire 1.

The pseudoelastic alloys include those with stress-strain curves due to tension in any shape, and those with notably measurable or unmeasurable transformation points such as, Af, Ms, and Mf, and all of those that are greatly deformed by stress but return mostly to the original shape when the stress is removed.

Examples of a preferred composition of the superelastic alloy include a Ni—Ti alloy such as Ni—Ti alloy of 49 to 52 atomic % Ni, a Cu—Zn alloy of 38.5 to 41.5 wt % Zn, a Cu—Zn—X alloy of 1 to 10 wt % X (X is at least one of Be, Si, Sn, Al, or Ga), a Ni—Al alloy of 36 to 38 atomic % Al, and the like. Among these, the above-mentioned Ni—Ti alloys are particularly preferable.

Further, a cobalt-based alloy can be used as the material of the wire body 2. When the wire body 2 is configured by the cobalt-based alloy, the medical guidewire 1 has particularly excellent torque transmissibility, and is extremely unlikely to suffer problems such as buckling. As the cobalt-based alloy, any cobalt-based alloy including Co as a constituent element may be used, but a cobalt-based alloy including Co as a main component (Co-based alloy: an alloy having the highest content in weight ratio of Co in the elements constituting the alloy) is preferably used, and a Co—Ni—Cr alloy is more preferably used. Using an alloy having such a composition makes the above-mentioned effect more remarkable. Further, an alloy having such a composition has a high elastic coefficient, can be cold-formed even at a high elastic limit, can form a reduced diameter while sufficiently preventing buckling due to the high elastic limit, and has sufficient flexibility and rigidity for insertion into a predetermined site.

Further, the wire body 2 may be configured by, for example, a piano wire instead of the above material.

Further, various forms can be adopted as a form of the wire body 2. For example, the wire body 2 may be formed with a single steel material, or may be formed by bending and twisting a single linear steel material. Further, the wire body 2 may be formed by twisting a plurality of linear steel materials, or may be formed by twisting a linear steel material and a linear resin member. Further, various configurations can be adopted, such as a configuration having a center and a surface formed of different materials (two-layer structure, for example, a member having an outer surface of the center including a metal, coated with a thermosetting resin to configure the surface). A total length of the wire body 2 is not particularly limited, but is preferably about 2,000 mm to 5,000 mm.

Further, the wire body 2 may be configured such that an outer diameter thereof is substantially constant, or a distal end thereof is formed in a tapered shape in which the outer diameter thereof decreases toward the distal end. When the distal end of the wire body 2 is formed in a tapered shape in which the outer diameter thereof decreases toward the distal end, the rigidity (bending rigidity and torsional rigidity) of the wire body 2 gradually decreases toward the distal end. As a result, the medical guidewire 1 can obtain a narrow segment having a good passage property and flexibility at the distal end and improved followability and safety, and can also prevent bending and the like, which is preferable.

Further, the wire body 2 may be configured by connecting a first wire body 2 configuring the distal end and a second wire body 2 configuring an intermediate portion and a hand-held portion, by welding or the like. When the wire body 2 is configured by the first wire body 2 and the second wire body 2, it is preferable to set a diameter of the first wire body 2 to be smaller than a diameter of the second wire body 2. Further, the connecting portion is preferably configured in a tapered shape to smoothly connect the first wire body 2 and the second wire body 2. When the wire body 2 is configured in this way, the rigidity (bending rigidity and torsional rigidity) of the wire body 2 gradually decreases toward the distal end. As a result, the medical guidewire 1 can obtain a narrow segment having a good passage property and flexibility at the distal end and improved followability and safety, and can also prevent bending and the like, which is preferable.

The intermediate layer 3 is configured to cover the surface of the wire body 2 and is configured by a material including a pigment and a binder resin. The pigment included in the intermediate layer 3 is a colorant and is used for coloring the intermediate layer 3. This pigment may be either an inorganic pigment or an organic pigment, but preferably has excellent heat resistance. As the pigment, carbon black, titanium oxide, phthalocyanine blue, mica, nickel titanium yellow, Prussian blue, Milori blue, cobalt blue, ultramarine, Viridian, and the like can be used. The pigment may be used alone or in combination (especially mixed) of two or more types. An average particle diameter of the pigment is not particularly limited, but is preferably set in a range of 0.05 μm or more and 2 μm or less, more preferably 0.1 μm or more and 1.5 μm or less.

Examples of the binder resin included in the intermediate layer 3, although a type thereof is not particularly limited, include polysulfone, polyimide, polyether ether ketone, polyarylene ketone, polyphenylene sulfide, polyarylene sulfide, polyamideimide, polyetherimide, polyimide sulfone, polyallyl sulfone, polyallyl ether sulfone, polyester, polyether sulfone, and the like. In particular, polyimide resins such as polyimide, polyamideimide, polyetherimide, and polyimide sulfone can be preferably used. Using such a material as the binder resin can effectively improve the adhesion between the wire body 2 and the outermost layer 4.

A thickness of the intermediate layer 3 is not particularly limited, but is preferably set to 1 μm or more in order to emphasize a colored tint. Further, in order to prevent the medical guidewire from being excessively thick, the thickness of the intermediate layer 3 is preferably set to 30 μm or less. More preferably, the intermediate layer 3 is configured in a range of 2 μm or more and 20 μm or less.

Further, in the present invention, a concentration of the pigment is configured to be in a range of 50 wt % or more and 90 wt % or less with respect to the intermediate layer 3 as a whole. More preferably, the concentration of the pigment is in a range of 55 wt % or more and 85 wt % or less with respect to the intermediate layer 3 as a whole, and still more preferably in a range of 60 wt % or more and 85 wt % or less. The concentration of the pigment set in this way increases a surface area of the surface of the intermediate layer 3 (surface in contact with the outermost layer 4), increases an anchor effect for the outermost layer 4, and dramatically improves adhesion strength of the outermost layer 4 to the intermediate layer 3. Further, the concentration of the pigment set in this way can reduce a hardness gap between the wire body 2 and the intermediate layer 3, and this makes it difficult for shear stress applied from outside of the guidewire to concentrate on an interface between the wire body 2 and the intermediate layer 3, for example, when the guidewire is passed through the distal end of the puncture needle.

A method of forming the intermediate layer 3 by coating the surface of the wire body 2 with the material configured by the pigment and the binder resin is not particularly limited, but various methods can be used. For example, the intermediate layer 3 can be formed by applying a solution prepared by mixing the pigment and the binder resin described above with an appropriate solvent to the wire body 2 and then drying the solution to volatilize the solvent. The material included in the intermediate layer 3 is not limited to the pigment and the binder resin described above, but may include, for example, a fluorine resin or various other additives.

The outermost layer 4 is configured to cover the intermediate layer 3 disposed on the surface of the wire body 2, and is preferably formed of a transparent material. As a material forming the outermost layer 4, for example, a fluororesin material having lubricity is preferable. Examples of such a fluororesin material include tetrafluoroethylene-perfluoroalkyl vinyl ether copolymer (PFA, melting point of 300° C. to 310° C.), polytetrafluoroethylene (PTFE, melting point of 330° C.), tetrafluoroethylene-hexa fluoropropylene copolymer (FEP, melting point of 250° C. to 280° C.), ethylene-tetrafluoroethylene copolymer (ETFE, melting point of 260° C. to 270° C.), polyvinylidene fluoride (PVDF, melting point of 160° C. to 180° C.), polychlorotrifluoro ethylene (PCTFE, melting point of 210° C.), tetrafluoroethylene-hexafluoropropylene-perfluoroalkyl vinyl ether copolymer (EPE, melting point of 290° C. to 300° C.), and copolymers including these polymers. Among these, PFA, PTFE, FEP, ETFE, and PVDF, which have excellent sliding properties, are preferable.

A thickness of the outermost layer 4 is not particularly limited, but a dry thickness thereof is usually 2 μm or more and 30 μm or less, preferably 3 μm or more and 25 μm or less, and particularly preferably 4 μm or more and 20 μm or less.

A method of forming the outermost layer 4 by coating the surface of the intermediate layer 3 with the above resin material is not particularly limited, and various methods can be used. In an example of such a method, the wire body 2 on which the intermediate layer 3 is formed is immersed in the solution prepared by using the above resin material and a suitable solvent and dried, and then heat treatment is performed to fuse the outermost layer 4 onto the intermediate layer 3. The heat treatment can be performed, for example, by using a chamber-type heat treatment device and applying heat from the outside of the outermost layer 4 formed on the wire body 2. Further, when the wire body 2 is formed of, for example, a metal material that easily conducts electricity, the wire body 2 is heated by applying a voltage to both ends of the wire body 2, and the outermost layer 4 can be fused using the heat onto the intermediate layer 3 by melting the outermost layer 4 disposed to cover the surface of the wire body 2.

The medical guidewire 1 according to the present embodiment is configured such that the intermediate layer 3 is interposed between the wire body 2 and the outermost layer 4 as described above, and the concentration of the pigment included in the intermediate layer 3 is 50 wt % or more and 90 wt % or less with respect to the intermediate layer 3 as a whole. Such a configuration can significantly enhance the adhesion between the outermost layer 4 and the intermediate layer 3, and can effectively prevent the outermost layer 4 from peeling off due to contact with the distal end of a hollow puncture needle, for example, when the guidewire 1 is used as a guide wire passed through the puncture needle in a ultrasonic endoscopic paracentesis.

Further, the medical guidewire 1 of the present invention, which includes the outermost layer 4 having a lubricity and formed of a fluororesin material or the like, exhibits extremely high smoothness and can ensure good slidability between an inner wall of a puncture needle and an inner wall of a catheter in an ultrasonic endoscopic paracentesis.

Here, in a configuration shown in FIG. 1, the intermediate layer is provided as a single layer. However, for example, as shown in an enlarged plan view of a main part in FIG. 2(a), and an enlarged sectional view of a main part taken along line A-A of FIG. 2(a) in FIG. 2(b), the intermediate layer may include a first region 31 including a first pigment and a second region 32 including a second pigment having a color different from that of the first pigment. In the medical guidewire shown in FIGS. 2(a) and 2(b), the second region 32 is provided on the first region 31 such that the intermediate layer has a two-layer structure. The second region 32 is provided on the first region 31 so as to form a spiral pattern along a longitudinal direction of the medical guidewire.

In this intermediate layer 3, for example, the first pigment, a binder resin, and a solvent are mixed to prepare a first solution, and separately, the second pigment, a binder resin, and a solvent are mixed to prepare a second solution. Then, after the first solution is applied to the wire body 2 and dried to form the first region 31, the second solution is spirally applied onto the first region 31 and dried to form the second region 32.

In the intermediate layer 3 having the first region 31 and the second region 32 as described above, the above-described effect of improving the adhesion can be obtained by setting the concentration of the pigment included in the intermediate layer 3 (the concentration of the pigment obtained by adding the first pigment and the second pigment) to be 50 wt % or more and 90 wt % or less with respect to the intermediate layer 3 as a whole. Further, the first region 31 and the second region 32 having different colors from each other are provided, and thus a movement of the guidewire can be easily recognized through an endoscopic fiberscope. The medical guidewire shown in FIG. 2 therefore has excellent visibility.

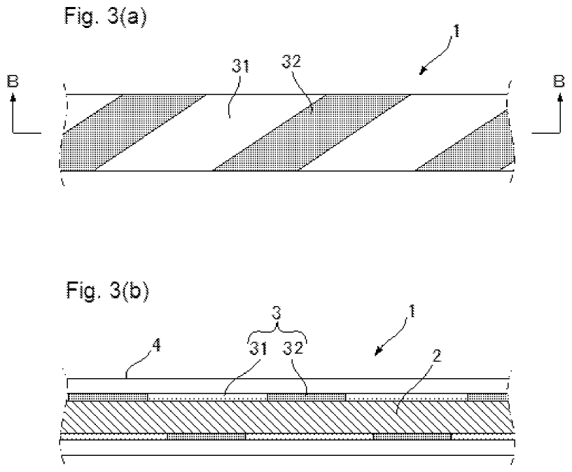

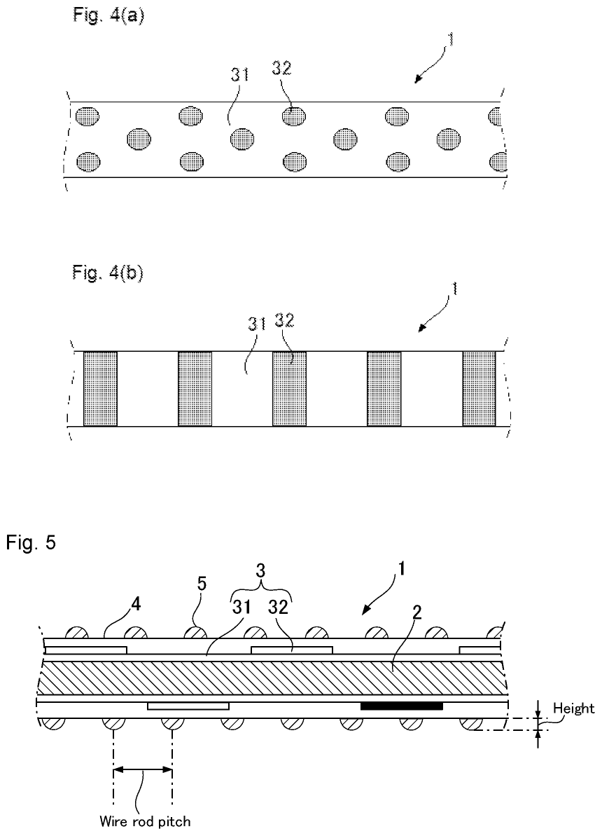

Further, FIGS. 2(a) and 2(b) show the structure of the intermediate layer 3 having a two-layer structure in which the second region 32 is formed as a spiral pattern on the first region 31. However, as shown in an enlarged plan view in FIG. 3(a) and an enlarged cross-sectional view taken along line B-B of FIG. 3(a) in FIG. 3(b), the first region 31 and the second region 32 may be configured as a double spiral structure (a form in which a spiral pattern is formed by the intermediate layer 3 as a single layer) disposed alternately on the wire body 2 along the longitudinal direction of the wire body 2. Further, when the first region 31 and the second region 32 having different colors from each other are provided, the configuration is not limited to the configuration in which a spiral pattern is formed as shown in FIGS. 2(a), 2(b), and 3. However, for example, the second region 32 may be formed in a dot shape as shown in an enlarged plan view of a main part in FIG. 4(a), or the ring-shaped first region 31 and the second region 32 may be alternately arranged along the longitudinal direction of a wire member as shown in an enlarged plan view of a main part in FIG. 4(b).

The inventors of the present invention created prototypes according to Examples (Examples 1 to 4) and Comparative Examples (Comparative Examples 1 to 4) relating to the medical guidewire of the present invention, and conducted a test for confirming the above-mentioned effects (effect of improved adhesion), which will be described below.

First, structures of Examples 1 to 4 and Comparative Examples 1 to 4 will be described. In Examples 1 to 4 and Comparative Examples 1 to 4, as shown in FIGS. 2(a) and 2(b), the intermediate layer 3 (intermediate layer 3 including the first region 31 and the second region 32) having a two-layer structure is formed on the wire body 2, and the outermost layer 4 is formed on the intermediate layer 3 that has been formed. In each of Examples 1 to 4 and Comparative Examples 1 to 4, a metal wire rod having a diameter of 0.55 mm (material: Ni—Ti alloy manufactured by Furukawa Techno Material Co., Ltd.) is used as the wire body 2. Further, as the outermost layer 4, in each of Examples 1 to 4 and Comparative Examples 1 to 4, a tetrafluoroethylene-perfluoroalkylvinylether copolymer (PFA) is used. The outermost layer 4 has a thickness of 10 μm.

A thickness of the first region 31 configuring the intermediate layer 3 is 4 μm. A thickness of the second region 32 formed on the first region 31 is 8 μm. Further, the second region 32 is configured such that a dimension along the longitudinal direction of the wire body 2 is 3 mm. Further, when viewed in the direction along the longitudinal direction of the wire body 2, an interval between the adjacent second regions 32 is 6 mm.

In Examples 1 to 4 and Comparative Examples 1 to 4, the intermediate layer 3 was formed by variously changing the pigment and the binder resin included in the first region 31 and the second region 32 and by changing the concentration of the pigment with respect to the intermediate layer 3 as a whole (first region 31 and second region 32). Details of the types of binder resins, the types of pigments, and the concentration of the pigment included in the intermediate layer 3 (first region 31 and second region 32) are shown on Table 1 below. The concentration of the pigment in Example 1 is 85 wt %, the concentration of the pigment in Example 2 is 70 wt %, the concentration of the pigment in Example 3 is 60 wt %, and the concentration of the pigment in Example 4 is 50 wt %. Further, the concentration of the pigment in Comparative Example 1 is 30 wt %, the concentration of the pigment in Comparative Example 2 is 40 wt %, the concentration of the pigment in Comparative Example 3 is 95 wt %, and the concentration of the pigment in Comparative Example 4 is 40 wt %.

TABLE 1

| | Intermediate layer | | | | | |
|---|---|---|---|---|---|---|
| | First region | | | Second region | | |
| | | | Concentration | | | Concentration |
| | Binder | Type of | of pigment | Binder | Type of | of pigment |
| | resin | pigment | (wt %) | resin | pigment | (wt %) |
| Example 1 | PAI | CB | 85 | PAI | Titanium | 85 |
| | | | | | oxide | |
| Example 2 | PEI | Titanium | 70 | PEI | Cobalt | 70 |
| | | oxide | | | blue | |
| Example 3 | PI | Titanium | 60 | PI | Cobalt | 60 |
| | | oxide | | | blue | |
| Example 4 | PEI | CB | 50 | PEI | Titanium | 50 |
| | | | | | oxide | |
| Comparative | PAI | CB | 30 | PAI | Titanium | 30 |
| Example 1 | | | | | oxide | |
| Comparative | PAI | CB | 40 | PAI | Titanium | 40 |
| Example 2 | | | | | oxide | |
| Comparative | PEI | CB | 95 | PEI | Titanium | 95 |
| Example 3 | | | | | oxide | |
| Comparative | PEI | Titanium | 40 | PEI | Cobalt | 40 |
| Example 4 | | oxide | | | blue | |

Note:
PAI: polyamideimide, PEI: polyetherimide, PI: polyimide, CB: carbon black

Each of the guidewires in Examples 1 to 4 and Comparative Examples 1 to 4 configured as described above was passed through a hollow puncture needle actually used in an ultrasonic endoscopic paracentesis (NEOLUS (1.20×38 mm) manufactured by Terumo Corporation) to perform an adhesion confirmation test as to whether the outermost layer 4 peeled off. More specifically, each guidewire was inserted from a base end of the hollow puncture needle placed horizontally, each guidewire was pulled out by 50 mm at an angle of 45 degrees upward from the distal end of the puncture needle and installed, and each guidewire was pulled from the distal end toward the base end of the puncture needle at a constant speed. Whether the outermost layer 4 peeled off from the guidewire was confirmed using a microscope. The results are shown on Table 2 below. In adhesion confirmation results, “poor” is given when the outermost layer 4 peeled off and fell off, and “fair” is given when the outermost layer 4 did not fall off but peeled off in layers, “good” is given when the outermost layer 4 was flawed but did not peel off or fall off, and “excellent” is given when the outermost layer 4 was not flawed or did not peel off.

The inventors also conducted a test for confirming the visibility of each of Examples 1 to 4 and Comparative Examples 1 to 4, and the results are also shown on Table 2. The test for confirming the visibility was performed by inserting each guidewire into a PTFE cannula and observing the movement of each guidewire through an endoscopic fiberscope. On Table 2, in the test confirmation results, “poor” indicates that the movements of the guidewires were not confirmed, and “fair” indicates that the movements of the guidewires were confirmed but unclear. Also, “excellent” indicates that the movement of the guidewires was clearly confirmed.

TABLE 2

| | Intermediate layer | Visibility | Adhesion |
|---|---|---|---|
| | Concentration of | confirmation | confirmation |
| | pigment (wt %) | results | results |
| Example 1 | 85 | Excellent | Excellent |
| Example 2 | 70 | Excellent | Excellent |
| Example 3 | 60 | Excellent | Good |
| Example 4 | 50 | Excellent | Fair |
| Comparative Example 1 | 30 | Fair | Poor |
| Comparative Example 2 | 40 | Fair | Poor |
| Comparative Example 3 | 95 | Excellent | Fair to poor |
| Comparative Example 4 | 40 | Fair | Poor |

As shown in the adhesion confirmation results on Table 2, in Examples 1 to 4 and Comparative Example 3 where the concentration of the pigment included in the intermediate layer 3 (the first region 31 and the second region 32) is in a range of 50 wt % or more with respect to the intermediate layer 3 as a whole, it can be confirmed that the outermost layer 4 did not peel off or fall off. In particular, in Examples 1 to 3 where the concentration of the pigment is in a range of 60 wt % or more and 85 wt % or less with respect to the intermediate layer 3 as a whole, it can be seen that none of the outermost layer 4 peeled off or fell off, and the adhesion of the outermost layer 4 was extremely excellent.

On the other hand, in Comparative Example 1, Comparative Example 2, and Comparative Example 4 where the concentration of the pigment is 40 wt % or less with respect to the intermediate layer 3 as a whole, it was confirmed that all of the outermost layer 4 partly fell off, and the adhesion of the outermost layer 4 was poor.

Further, from the adhesion confirmation results on Table 2, it is considered that a lower limit of the concentration of the pigment at which the outermost layer can withstand the use without falling off is 50 wt % as in Example 4. Further, in particular, the lower limit of the concentration of the pigment at which the adhesion of the outermost layer 4 becomes high is considered to be between 50 wt % as in Example 3 and 60 wt % as in Example 4, and it is estimated that 55 wt % as an arithmetic mean value of both values is a boundary. Therefore, in order to sufficiently secure the adhesion between the intermediate layer 3 and the outermost layer 4, the concentration of the pigment included in the intermediate layer 3 is preferably set to 55 wt % or more with respect to the intermediate layer 3 as a whole.

Further, from the adhesion confirmation results on Table 2, the upper limit of the concentration of the pigment at which the outermost layer can withstand use without falling off is considered to be 95 wt % as in Comparative Example 3. In Comparative Example 3, there may be a problem in the adhesion between the wire body 2 and the intermediate layer 3 due to an excessive amount of pigment included in the intermediate layer 3 (the adhesion confirmation result on Table 2 is “fair to poor” because there may be a problem in the adhesion between the wire body 2 and the intermediate layer 3). Therefore, an upper limit of the concentration of the pigment at which the adhesion between the wire body 2 and the intermediate layer 3 is excellent, and the adhesion of the outermost layer 4 is high is considered to be between 85 wt % as in Example 1 and 95 wt % as in Comparative Example 3. It is considered that 90 wt % as the arithmetic mean value of both values is a boundary. That is, in order to sufficiently secure the adhesion between the wire body 2 and the intermediate layer 3 and the adhesion between the intermediate layer 3 and the outermost layer 4, the concentration of the pigment included in the intermediate layer 3 is preferably set to 90 wt % or less with respect to the intermediate layer 3 as a whole.

Further, from visibility confirmation results on Table 2, in Examples 1 to 4 and Comparative Example 3 where the concentration of the pigment included in the intermediate layer 3 (the first region 31 and the second region 32) is in a range of 50 wt % or more with respect to the intermediate layer 3 as a whole, it can be seen that the movement of the guidewires can be clearly confirmed and the guidewires have good visibility. On the other hand, in Comparative Example 1, Comparative Example 2, and Comparative Example 4 where the concentration of the pigment is 40 wt % or less with respect to the intermediate layer 3 as a whole, the movement of the guidewire was not confirmed or was unclear.

As described above, setting the concentration of the pigment included in the intermediate layer 3 (the first region 31 and the second region 32) in a range of 50 wt % or more and 90 wt % or less with respect to the intermediate layer 3 as a whole can make the visibility through the endoscopic fiberscope good, and further secure the sufficient adhesion of the outermost layer 4.

Although the medical guidewire 1 of the present invention has been described above, the specific configuration is not limited to the above embodiment. For example, as shown in the sectional view in FIG. 5, a wire rod 5 may be spirally wound around the surface of the outermost layer 4 to form the medical guidewire 1. The medical guidewire 1 shown in FIG. 5 has a configuration in which the wire rod 5 is wound around the medical guidewire shown in FIG. 2. This wire rod 5 is preferably formed of the same material as the material forming the outermost layer 4. Further, the wire rod 5 is formed to have a substantially uniform thickness along the longitudinal direction thereof before being wound around the outermost layer 4. A maximum diameter of the wire rod 5, for example, in a range of 10 μm or more and 200 μm or less can be suitably used, and the maximum diameter of the wire rod 5 is preferably in a range of 80 μm or more and 200 μm or less. Here, a pitch is a concept representing a center-to-center distance between adjacent wire rods 5 in the direction along the longitudinal direction of the wire body 2 as shown in the sectional view in FIG. 5. In FIG. 5, the wire rod 5 is spirally wound such that each center-to-center distance (pitch) between adjacent wire rods 5 is equal. The center-to-center distance (pitch) between the wire rods 5 can be set to have an arbitrary dimension, and is, for example, from 15 μm to 5000 μm, preferably from 30 μm to 1000 μm, and particularly preferably from 50 μm to 700 μm. The center-to-center distance (pitch) between the wire rods 5 may be partially different.

A method of winding the wire rod 5 around the outermost layer 4 is not particularly limited, but examples of the method include a method of winding using a covering device used for producing a covering yarn.

Further, the wire rod 4 spirally wound around the outermost layer 4 is entirely fused onto and integrated with the outermost layer 4. Examples of the method of fusing the wire rod 5 onto the outermost layer 4 include a method in which the wire rod 5 is spirally wound around an outer surface of the outermost layer 4, and then the wire rod 5 and the outermost layer 4 are melted by heating, and the wire rod 5 is fused onto the surface of the outermost layer 4. As a method of heating, for example, heat is applied from the outside of the wire rod 4 wound around the outermost layer 4 on the wire body 2, using a chamber-type heat treatment device. Further, when the wire body 2 is formed of, for example, a metal material that easily conducts electricity, the wire body 2 is heated by applying a voltage to both ends of the wire body 2, and the wire rod 5 can be fused using the heat onto the outermost layer 4 by melting the outermost layer 4 and the wire rod 5 on the wire body 2. When the wire rod 5 is provided on the outermost layer 4, the heat treatment during a formation of the outermost layer 4 on the intermediate layer 3 may be omitted, and the outermost layer 4 may be fused to the intermediate layer 3 and the wire rod 5 may be fused to the outermost layer 4 at the same time by the heat treatment performed after the wire rod 5 is disposed on the outermost layer 4.

Provided with such wire rod 5, the outermost layer 4 has further improved durability, and when the medical guidewire 1 is inserted into a hollow puncture needle or a catheter, a portion in contact with the inner wall of the hollow puncture needle or the like is an outermost part (top) of the wire rod 5. Thus, it is possible to reduce a contact area between the medical guidewire 1 and the hollow puncture needle, catheter, or the like, and to secure higher slidability. In particular, configuring the wire rod 5 by a fluororesin material can secure higher slidability.

Further, the wire rod 5 thermally fused onto the outermost layer 4 has a section in a semi-cylindrical lens shape or a plano-convex lens shape (English capital letter “D” shape) as shown in the sectional view in FIG. 5. However, a height of the wire rod 5 after thermal fusion (dimension from the surface of the outermost layer 4 to a top of the wire rod) is preferably in a range of 4 μm to 80 μm. Configuring the wire rod 5 that has been fused to have such a numerical range, particularly 6 μm or more in height improves the smoothness due to a point contact when the medical guidewire 1 is moved inside the hollow puncture needle or catheter. Further, a vibration caused by a movement of an irregular portion is transmitted to the fingertip of the user (operator) of the medical guidewire, and thus the user can recognize a state of insertion from sensory information due to such a peculiar vibration in addition to an endoscopic visual information and normal insertion sensory information, thereby improving convenience of the user.

In the configuration shown in FIG. 5, one wire rod 5 is spirally wound around the outermost layer 4, but for example, two wire rods 5 having different thicknesses may be wound around the outermost layer 4 in a spiral shape (in a double spiral shape).

### DESCRIPTION OF REFERENCE SIGNS

1 medical guidewire
2 wire body
3 intermediate layer
31 first region
32 second region
4 outermost layer
5 wire rod

1. A guide wire comprising:
a wire body having a long outer shape;
an intermediate layer having a first region and a second region and covering the wire body; and
a resin layer with optical transparency and covering the intermediate layer, the resin layer having an irregular portion on a surface, the irregular portion having recesses and projections formed alternately along a longitudinal direction of the wire body in a cross section along the longitudinal direction,
wherein
the recesses and the projections are formed of a same material as each other,
the first region and the second region have different colors from each other,
in the intermediate layer, the first region and the second region appear alternately along the longitudinal direction, and
in a cross section along the longitudinal direction, a width of the second region is wider than a width of each projection.

2. The guide wire according to claim 1, wherein in the cross-section along the longitudinal direction, the projections have a semi-cylindrical lens shape or a plano-convex lens shape.

3. The guide wire according to claim 1, wherein in the longitudinal direction, a pitch of the second region is different from a pitch of the projections.

4. The guide wire according to claim 1, wherein a boundary between the first region and the second region is formed flat.

5. The guide wire according to claim 2, wherein in the longitudinal direction, a pitch of the second region is different from a pitch of the projections.

6. The guide wire according to claim 2, wherein a boundary between the first region and the second region is formed flat.

7. The guide wire according to claim 3, wherein a boundary between the first region and the second region is formed flat.

8. The guide wire according to claim 5, wherein a boundary between the first region and the second region is formed flat.
